(19) United States
(12) Patent Application Publication
(10) Pub. No.: US 2021/0161272 A1
(43) Pub. Date: Jun. 3, 2021

(54) MANICURE DEVICE

(71) Applicant: COSMEX CO. LTD., New Taipei City (TW)

(72) Inventors: Wan-Chieh HSIEH, New Taipei City (TW); Ya-Wen WU, New Taipei City (TW); Wen-Shan CHUNG, New Taipei City (TW); Yu-Ching LI, New Taipei City (TW)

(21) Appl. No.: 17/105,784

(22) Filed: Nov. 27, 2020

(30) Foreign Application Priority Data
Nov. 29, 2019 (TW) 108215949

(51) Int. Cl. A45D 29/00; G06F 3/01; H04W 4/80; A61B 5/0205

(52) U.S. Cl. CPC A45D 29/00; G06F 3/017; A61L 2/10; A61B 5/0205; H04W 4/80

(57) ABSTRACT

The present invention relates to a manicure device including at least one sensor to make it easier for a user to know the working mode and operating conditions of the manicure device. Moreover, the manicure device of the invention can operate under automatic control in order to have its working mode and operating conditions adjusted or set, or to issue a notification signal, after the working mode and operating conditions of the manicure device are sensed; therefore, it is convenient for a user to use the manicure device.

### BACKGROUND OF THE INVENTION

### 1. Technical Field

The present invention relates to a manicure device and more particularly to a manicure device with at least one sensor.

### 2. Description of Related Art

In recent years, with the advancement of smart technology, smart technology has begun to get involved in beauty-related products and markets, and manicure devices are no exception. Many companies have begun to try to apply smart technology to the manicure device and add many peripheral functions so that the manicure device has more than a general nail painting function. For example, using a photographic display to photograph the hand and preview the manicure result before performing the manicure, printing photos or patterns on the nail with manicure method, or recording and sharing painted nail photos on a social network.

In addition to providing customized services, a smart manicure device allows to preview an intended painting nails before manicuring, which can reduce errors happening when a manicurist is manicuring customers' nails, reduce customer complaint cases, and reduce the cost and time for customers to rebuilt manicure.

What's more, many smart manicure devices were so developed that they can be used at home to complete the painting of nails in the absence of a professional manicurist. Such a domestic smart manicure device includes preset nail painting patterns, so a user only has to put their fingers into the device in order to have the fingernails painted, thereby satisfying the need to beautify the nails in a cost-effective, time-saving, and do-it-yourself (DIY) manner. While the nail painting results of the aforesaid manicure devices are slightly different from those produced by a manicurist in terms of delicacy (e.g., with regard to the application of rhinestones or sequins), the majority of manicure enthusiasts who only desire basic nail painting (e.g., those who paint their nails in a single color only) can achieve what they want with those manicure devices.

### BRIEF SUMMARY OF THE INVENTION

Although smart technology has been applied to manicure devices, its functions are generally limited to providing a preview of an intended nail painting result and sharing painted nail photos on a social network. In other words, the development of smart technology in the field of manicure is still quite limited. The present invention provides a manicure device that has one or more sensors for sensing the use conditions of the manicure device effectively so as to provide a diversity of functions and services.

That is, the objective of the present invention is to provide a manicure device, comprising a manicure device body, at least one sensor, and a controller. The manicure device body includes a housing, an internal cavity, and at least one ultraviolet (UV) light-emitting unit, wherein the internal cavity is provided at one side of the housing and forms an opening at the housing, and the UV light-emitting unit is provided in the housing and is configured to emit light toward the internal cavity. The sensor is provided at the housing and is configured to send out a signal when triggered. The controller is provided on or in the housing, wherein the controller is connected to the sensor and is configured to receive the signal of the sensor.

In a preferred embodiment, the manicure device further includes an input interface configured to generate a setting signal through inputting, and the setting signal is sent to the controller, in order for the controller to perform a control procedure according to the setting signal.

In a preferred embodiment, the sensor includes a hand gesture sensor, a photosensor, a distance sensor, an motion sensor, a magnetic field sensor, a gyroscope, a GPS (Global Positioning System) device, a heart rate sensor, a UV light sensor, a temperature sensor, a chemical substance sensor, a radio frequency identification (RFID) device, a dust sensor, a position detector, a virtual image key sensor or an infrared sensor.

In a preferred embodiment, the sensor is a hand gesture sensor, the signal corresponds to a hand gesture feature, and the hand gesture sensor extracts the hand gesture feature from an area of interest of an image and sends the hand gesture feature to the controller, in order for the controller to output a control procedure according to the hand gesture feature.

In a preferred embodiment, the hand gesture sensor is an ultrasound sensing device or an infrared sensing device.

In a preferred embodiment, the sensor is a photosensor, the signal corresponds to a lighting parameter, and the photosensor obtains the lighting parameter from the light in an environment and sends the lighting parameter to the controller, in order for the controller to output a control procedure according to the lighting parameter.

In a preferred embodiment, the sensor is a distance sensor, the signal corresponds to a distance parameter, and the distance sensor obtains the distance parameter by sensing a distance to a target object and sends the distance parameter to the controller, in order for the controller to output a control procedure according to the distance parameter.

In a preferred embodiment, the sensor is an motion sensor, the signal corresponds to an acceleration parameter, velocity parameter, or displacement parameter, and the motion sensor obtains the acceleration parameter, velocity parameter, or displacement parameter by sensing motion changes in an environment and sends the acceleration parameter, velocity parameter, or displacement parameter to the controller, in order for the controller to output a control procedure according to the acceleration parameter, velocity parameter, or displacement parameter.

In a preferred embodiment, the sensor is a magnetic field sensor, the signal corresponds to a magnetic field parameter, and the magnetic field sensor obtains the magnetic field parameter by sensing a state of a magnetic field in an environment and sends the magnetic field parameter to the controller, in order for the controller to output a control procedure according to the magnetic field parameter.

In a preferred embodiment, the sensor is a gyroscope, the signal corresponds to an orientation parameter, and the gyroscope obtains the orientation parameter by sensing an orientation of an environment and sends the orientation parameter to the controller, in order for the controller to output a control procedure according to the orientation parameter.

In a preferred embodiment, the sensor is a GPS (Global Positioning System) device, the signal corresponds to a geographic location parameter, and the GPS device obtains the geographic location parameter by sensing a geographic location of an environment and sends the geographic location parameter to the controller, in order for the controller to output a control procedure according to the geographic location parameter.

In a preferred embodiment, the sensor is a heart rate sensor, the signal corresponds to a heart rate parameter, and the heart rate sensor obtains the heart rate parameter by sensing a heart rate of a living creature in an environment and sends the heart rate parameter to the controller, in order for the controller to output a control procedure according to the heart rate parameter.

In a preferred embodiment, the sensor is a UV light sensor, the signal corresponds to a UV light parameter, and the UV light sensor obtains the UV light parameter by sensing a UV light in an environment and sends the UV light parameter to the controller, in order for the controller to output a control procedure according to the UV light parameter.

In a preferred embodiment, the sensor is a temperature sensor, the signal corresponds to a temperature parameter, and the temperature sensor obtains the temperature parameter by sensing a temperature and sends the temperature parameter to the controller, in order for the controller to output a control procedure according to the temperature parameter.

In a preferred embodiment, the sensor is a chemical substance sensor, the signal corresponds to a chemical substance parameter, and the chemical substance sensor obtains the chemical substance parameter by sensing the chemical substance in an environment and sends the chemical substance parameter to the controller, in order for the controller to output a control procedure according to the chemical substance parameter.

In a preferred embodiment, the sensor is a radio frequency identification (RFID) device, the signal corresponds to an RFID parameter, and the RFID device obtains the RFID parameter by receiving and/or transmitting an RFID and sends the RFID parameter to the controller, in order for the controller to output a control procedure according to the RFID parameter; and/or, an external RFID reader can identify RFID parameter of the RFID device in order for the external RFID reader to obtain specific information and/or output corresponding procedure according to the RFID parameter of the RFID device.

In a preferred embodiment, the sensor is a radio frequency identification (RFID) device, the signal corresponds to an RFID parameter, and the RFID device obtains the RFID parameter by receiving and/or transmitting an RFID parameter.

In a preferred embodiment, the sensor is a dust sensor, the signal corresponds to a dust parameter, and the dust sensor obtains the dust parameter by sensing the dust in an environment and sends the dust parameter to the controller, in order for the controller to output a control procedure according to the dust parameter.

In a preferred embodiment, the sensor is a position detector, the signal corresponds to a position parameter, and the position detector obtains the position parameter by sensing a state of an object in an area of interest and sends the position parameter to the controller, in order for the controller to output a control procedure according to the position parameter.

In a preferred embodiment, the sensor is a virtual image key sensor, the signal corresponds to a virtual image key parameter, and the virtual image key sensor obtains the virtual image key parameter by sensing a virtual image key in an environment and/or an altered image of the virtual image key and sends the virtual image key parameter to the controller, in order for the controller to output a control procedure according to the virtual image key parameter.

In a preferred embodiment, the virtual image key is generated by an image projector.

In a preferred embodiment, each of the ultraviolet (UV) light-emitting unit comprises at least one UV LED chip, and the UV LED chip has a full width half maximum equal or smaller than 30 nm.

In a preferred embodiment, the power consumption of the manicure device is smaller than 40 watts.

In a preferred embodiment, the manicure device comprises an image analysis module provided in an interior of the housing, wherein the image analysis module comprises a camera unit and an analysis unit, the camera unit shots a picture of an object, the analysis unit analysis the picture and sends result parameter to the controller, in order for the controller to output a control procedure according to the result parameter.

Unlike its prior art counterparts, the manicure device of the present invention has at least one sensor to make it easier for a user to know the working mode and operating conditions of the manicure device. Moreover, the manicure device of the invention can operate under manual or automatic control in order to have its working mode and operating conditions adjusted or set, or to issue a notification signal.

### BRIEF DESCRIPTION OF THE SEVERAL VIEWS OF THE DRAWINGS

FIG. 1 is a schematic drawing of the manicure device according to the first embodiment of the present invention.

FIG. 2 is a schematic drawing of the manicure device according to the second embodiment of the present invention.

FIG. 3 is a flowchart of the manually controlled operation of the manicure device of the present invention.

FIG. 4 is a flowchart of the automatically controlled operation of the manicure device of the present invention.

### DETAILED DESCRIPTION OF THE INVENTION

The details and technical solution of the present invention are hereunder described with reference to accompanying drawings. For illustrative sake, the accompanying drawings are not drawn to scale. The accompanying drawings and the scale thereof are not restrictive of the invention.

The following embodiments should not be regarded as excessively limiting the present invention. A person with ordinary knowledge in the technical field to which the invention pertains can modify and change the embodiments discussed herein without departing from the spirit or scope of the invention, and the variation and modification according to the invention may still fall into the scope of the invention.

As used herein, the terms “in” and “outside” generally make reference to the entirety of the manicure device of the present invention. When reference is made to the housing of the manicure device, “in” refers to being on the side that is relatively close to the internal cavity of the manicure device, and “outside” refers to being on the side that is relatively far from the internal cavity. For example, a controller that is locked to the housing (e.g., to an inner housing portion or an upper housing portion) or between the inner housing portion and the upper housing portion and is not directly exposed to view can be referred to herein as being “provided in the housing”.

As used herein, the terms “front” and “rear” generally make reference to the opening of the housing of the manicure device of the present invention. “Front” refers to being relatively close to the opening end whereas “rear” refers to being relatively far from the opening end.

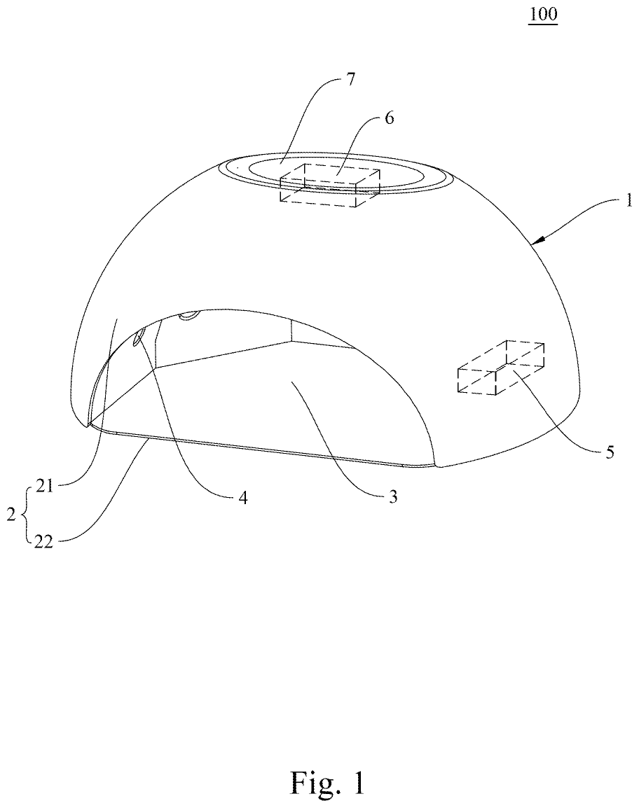

Please refer to FIG. 1 for a schematic drawing of the manicure device according to the first embodiment of the present invention. In this embodiment, the manicure device 100 includes a manicure device body 1, a sensor 5, and a controller 6. The manicure device body 1 includes a housing 2, an internal cavity 3 that is provided at one side of the housing 2 and forms an opening at the housing 2, and a plurality of ultraviolet (UV) light-emitting units 4 that are provided in the housing 2 and are configured to emit light toward the internal cavity 3. Each of the ultraviolet (UV) light-emitting unit 4 comprises at least one UV LED chip, and the UV LED chip has a full width half maximum equal or smaller than 30 nm. The sensor 5 is provided at the housing 2 and is configured to send out a signal when triggered; the sensor 5 is triggered by sensing a sensing signal from different types of parameter changes. A technician may adjust the sensor 5 according to application requirements in order to expose the sensor 5 (e.g., from the inner or outer side of the housing 2) or not to expose the sensor 5. The controller 6 is provided at the housing 2. More specifically, the controller 6 is coupled to an input interface 7 on the outer side of the housing 2 and is connected to the sensor 5 in order to receive the signal of the sensor 5.

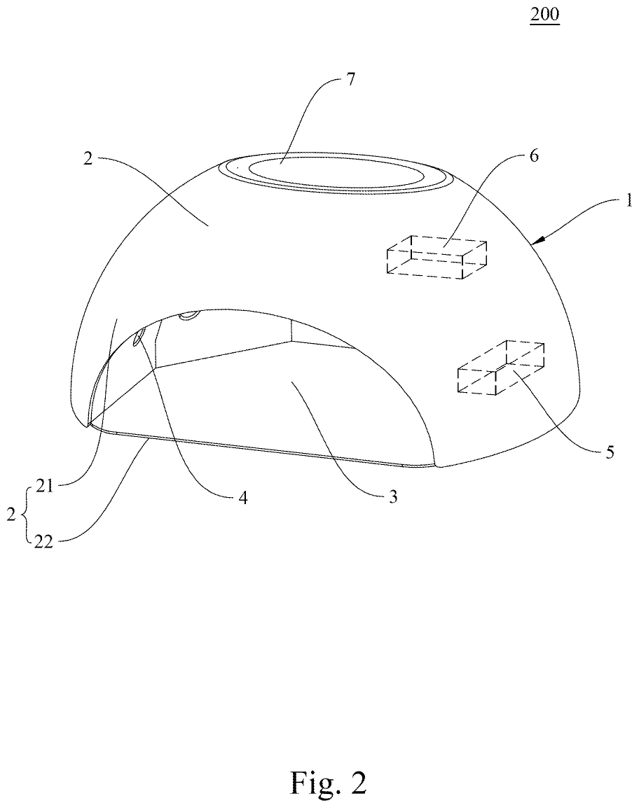

Please refer to FIG. 2 for a schematic drawing of the manicure device according to the second embodiment of the present invention. In this embodiment, the manicure device 200 includes the same manicure device body 1, sensor 5, controller 6, and input interface 7 as in the first embodiment, and is different from the first embodiment in that the controller 6 is provided in the housing 2, i.e., within the housing 2 itself.

The housing 2 includes an upper housing portion 21 and a base portion 22. The upper housing portion 21 and the base portion 22 may be integrally formed, fixedly coupled together, or separably coupled together; the present invention has no limitation in this regard. The controller 6 may be provided in the upper housing portion 21 or the base portion 22; the invention has no limitation in this regard either. More specifically, the housing 2 may further include an inner housing portion (not shown) provided on the inner side of the upper housing portion 21. The controller 6 may be provided on the upper housing portion 21, on the inner housing portion, or in the space between the upper housing portion 21 and the inner housing portion; a technician may adjust the location of the controller 6 to meet configuration requirements.

The sensor 5 may be provided at any component of the manicure device 100 or 200 as needed. For example, the sensor 5 may be provided in the housing (i.e., unexposed), or on the outer side of the upper housing portion 21 in order to sense a sensing signal outside the manicure device 100 or 200, or on the inner side of the upper housing portion 21 in order to sense a sensing signal corresponding to the internal cavity 3, or on the upper housing portion 21, or on the inner housing portion (if existing), or in the space between the upper housing portion 21 and the inner housing portion (if existing), or in the base portion 22 (i.e., unexposed), or on the base portion 22, or at a front outer end of the base portion 22 in order to sense a sensing signal corresponding to a front bottom side of the manicure device 100 or 200, or at a rear outer end of the base portion 22 in order to sense a sensing signal corresponding to a rear bottom side of the manicure device 100 or 200; the present invention has no limitation in this regard. The sensor 5 includes but is not limited to a hand gesture sensor, a photosensor, a distance sensor, an motion sensor, a magnetic field sensor, a gyroscope, a GPS (Global Positioning System) device, a heart rate sensor, a UV light sensor, a temperature sensor, a chemical substances sensor, a radio frequency identification (RFID) device, a dust sensor, a position detector and a virtual image key sensor.

The input interface 7 can be electrically or wirelessly connected to the controller 6. The input interface 7 can be a general operation interface for controlling an electronic device, such as a touch panel, a touch display panel, or a mechanical button, knob or push rod; the present invention has no limitation in this regard.

Each of the UV light-emitting units 4 of the present invention comprises at least one UV LED chip. For example, the UV light-emitting unit 4 is a UV LED bulb with one UV LED chip and one non-UV LED chip (such as visible-light LED chips). The amount of the UV LED chips and non-UV LED chips in each of the UV light-emitting unit 4 has no limitation.

The manicure device 100 or 200 of the present invention may be provided with other components as needed, such as a display device, a light-emitting device, a sound-playing device, a heat dissipation device, or an image projector; the invention has no limitation in this regard. For example, when the manicure device 100 or 200 is provided with a display device, a light-emitting device, or a sound-playing device, the display device or the sound-playing device can be used to notify a user of the working mode of the manicure device 100 or 200 and the operating condition (i.e., the sensing signal parameter) sensed by the sensor 5, or to display or play advertising information (e.g., scrolling text) or other images or sounds, and the light-emitting device can be provided on the housing of the manicure device 100 or 200 to produce notification light, alerting light, or cool fashionable light as a notification or alert or for entertainment or esthetic purposes.

The manicure device 100 or 200 of the present invention can optionally be coupled with other electronic devices, such as smart electronic products, computers, or wearable products; for example, tablet computers, smart phones, or smart watches; the invention has no limitation in this regard.

The power consumption of the manicure device 100 or 200 is smaller than 40 watts. The power consumption relates to the curing condition, such as curing time or temperature. Specifically, when the curing time is longer than 2 mins, the power consumption will exceed 40 watts, which discomforts users with high heat or even pain and injury and causes the gel nail polishes yellowed or wrinkled.

The manicure device 100 or 200 of the present invention may optionally comprise an image analysis module provided in an interior of the housing 2, wherein the image analysis module comprises a camera unit and an analysis unit, the camera unit shots a picture of an object, the analysis unit analysis the picture and sends result parameter to the controller, in order for the controller to output a control procedure according to the result parameter. The control procedure may involve providing suggestions for user to adjusting the position of fingers or toes, providing suggestion for user to put preferable decoration on the nail, adjusting the UV light-emitting units 4 angularly toward the fingers or the position of the UV light-emitting units 4, or adjusting the curing condition (such as curing time or temperature); the present invention has no limitation in this regards.

The manicure device 100 or 200 of the present invention may optionally be provided with other functional units in any component of the manicure device 100 or 200 or in an electronic device coupled with the manicure device 100 or 200, such as a recording unit or a storage unit; the invention has no limitation in this regard. For example, the recording unit and/or the storage unit are set in the controller 6, in the manicure device 100 or 200, and/or in an electronic device (such as a mobile phone or a tablet computer) coupled with the manicure device 100 or 200. When the sensor 5 receives a sensing signal, the sensing signal can be recorded in the recording unit and stored in the storage unit.

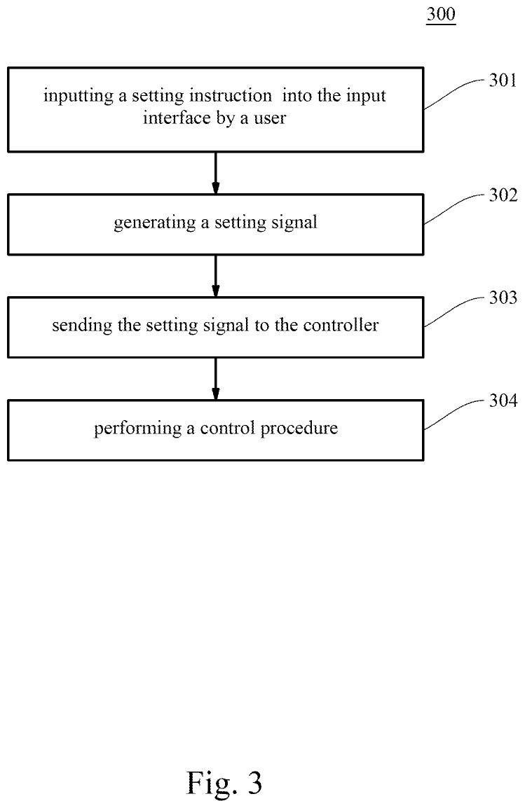

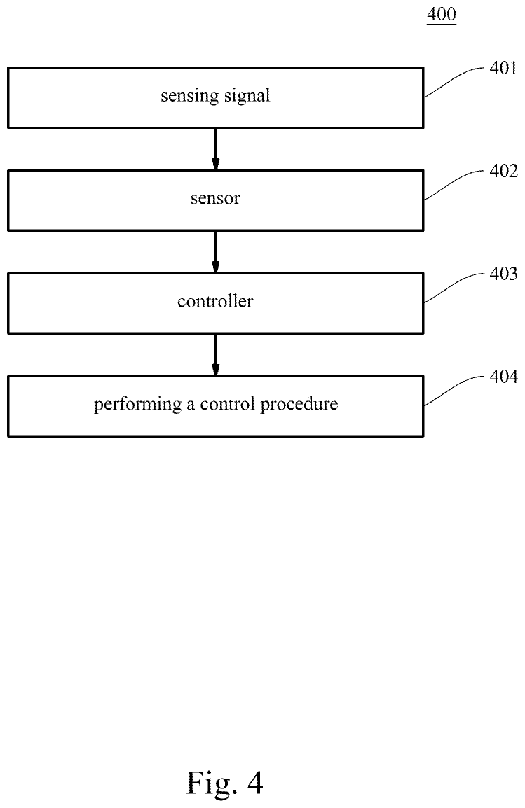

The operation mode of the manicure device 100 or 200 of the present invention can be manual control operation, automatic control operation, or both manual control operation and automatic control operation. The operation flow of the manual control operation and that of the automatic control operation are shown in FIG. 3 and FIG. 4 respectively.

Please refer to FIG. 3 for the flowchart of the manually controlled operation of the manicure device 100 or 200 of the present invention. The manually controlled operation 300 includes the steps of: inputting a setting instruction into the input interface 7 by a user (301), generating a setting signal (step 302), sending the setting signal to the controller 6 (step 303), and performing a control procedure by the controller 6 according to the setting signal (step 304) in order to set or adjust the manicure device 100 or 200.

When the manicure device 100 or 200 of the present invention is operating under manual control, the controller 6 receives the signal sensed by the sensor 5 and then instructs a display device, sound-playing device, or light-emitting device of the manicure device 100 or 200 to display, play, or emit a notification signal. A user, therefore, will be able to know the working mode and the operating conditions of the manicure device 100 or 200 by looking at the display device or by perceiving the notification signal otherwise, and then set or adjust the manicure device 100 or 200 through the manually controlled operation 300 stated above.

Please refer to FIG. 4 for the flowchart of the automatically controlled operation of the manicure device 100 or 200 of the present invention. The automatically controlled operation 400 includes the steps of: generating a sensing signal by the environment (step 401), triggering the sensor 5 (step 402), sending a signal to the controller 6 (step 403), and performing a control procedure by the controller 6 (step 404) in order to set, adjust, or otherwise control the manicure device 100 or 200.

Preferably, the manicure device 100 or 200 of the present invention is provided with a display device, a sound-playing device, or a light-emitting device when configured to operate under automatic control. The signal received by the controller 6 will trigger the display device, the sound-playing device, or the light-emitting device to display, play, or emit a notification signal so that a user can know the working mode and the operating conditions of the manicure device 100 or 200 by looking at the display device or by perceiving the notification signal otherwise. Apart from allowing the manicure device 100 or 200 to operate under automatic control, it is more preferable that a user can adjust or set the manicure device 100 or 200 by manual control after knowing the working mode and the operating conditions of the manicure device 100 or 200.

The step of “performing a control procedure” includes adjusting, turning on, or turning off the manicure device 100 or 200, any of its components or functional units, any electronic device that may be coupled to the manicure device 100 or 200 as needed, or any of the components or functional units of such electronic devices. For example, performing a control procedure may include turning on or off the manicure device 100 or 200; adjusting the UV light intensity or irradiation time of the UV light-emitting units 4; turning on or off the UV light-emitting units 4; adjusting the temperature of the internal cavity 3; turning on the recording unit and/or storage unit of the manicure device 100 or 200 and, if necessary, transmitting the recorded or stored data to an electronic device (e.g., a mobile phone or tablet computer) coupled to the manicure device 100 or 200; turning on a display device, light-emitting device, or sound-playing device of the manicure device 100 or 200 in order to issue a notification, e.g., to notify the user of the working state of the manicure device 100 or 200 or alert the user with an image, sound, or flashing light; or turning on or off an additional heat dissipation device of the manicure device 100 or 200; the present invention has no limitation in this regard.

The term “environment” includes the outer periphery of the housing 2 of the manicure device 100 or 200, or the interior of the internal cavity 3.

The hand gesture sensor is configured to sense a sensing signal and send out a signal, and the sensing signal and the signal correspond to a hand gesture feature. The hand gesture sensor extracts a hand gesture feature from the area of interest of an image and sends the hand gesture feature to the controller 6, in order for the controller 6 to output a control procedure according to the hand gesture feature. The term “the area of interest of an image” indicates that the hand gesture sensor will only extract the hand gesture feature in a hand gesture feature-containing area, i.e., the area of interest, of an image captured by the hand gesture signal sensor of the environment. For example, when sensing the hand gesture feature of “five fingers spread apart” in the internal cavity 3 or at a position 5 cm above and in front of the housing 2, the hand gesture sensor captures an image, extracts the hand gesture feature of “five fingers spread apart”, and sends the hand gesture feature to the controller 6. If the hand gesture feature of “five fingers spread apart” corresponds to the control procedure of turning on the UV light-emitting units 4, the controller 6 will, upon receiving and according to the hand gesture feature of “five fingers spread apart”, output the control procedure of turning on the UV light-emitting units 4. Using hand gesture features to control the manicure device 100 or 200 helps reduce not only the material cost required for the provision of physical press-keys, but also problems associated with physical press-keys (e.g., coming off or having poor contact after long-term use), thus extending the service life of the manicure device 100 or 200.

The hand gesture feature/hand gesture sensor may be any sensing device capable of detecting hand gesture features, such as an ultrasound sensing device, an infrared sensing device, or an image capture device; the present invention has no limitation in this regard.

The term “hand gesture feature” refers to dynamic as well as static hand gestures. Static hand gestures include, but are not limited to, the following gestures of a hand: a fist, the five fingers spread out, one finger extended out while the other four fingers are clenched (e.g., only the thumb or the index finger is extended out), two fingers extended out while the other three fingers are clenched (e.g., the index finger and the middle finger are extended out, or the thumb and the little finger are extended out), three fingers extended out while the other two fingers are clenched (e.g., the index finger, the middle finger, and the ring finger are extended out), and four fingers extended out while the other finger is clenched (e.g., the index finger, the middle finger, the ring finger, and the little finger are extended out). Dynamic hand gestures include various dynamic instructions made by moving at least one finger or the palm of a hand, such as but not limited to moving the index finger in a single direction (e.g., upward, downward, leftward, or rightward), moving the thumb and the index finger apart from a mutually touching state, drawing a circle with the index finger, writing with a finger, and spreading or closing the palm.

Preferably, the manicure device 100 or 200 is provided with the hand gesture feature sensor, and the hand gesture feature sensor can supersede the input interface 7, i.e., a user can input a setting instruction directly through the hand gesture feature sensor.

The photosensor is configured to sense a sensing signal and send out a signal, and the sensing signal and the signal correspond to a lighting parameter. The photosensor obtains the lighting parameter from the light in the environment and sends the lighting parameter to the controller 6, in order for the controller 6 to output a control procedure according to the lighting parameter. Preferably, the ambient light from which the photosensor obtains the lighting parameter is mainly light outside the housing 2 of the manicure device 100 or 200. For example, when the manicure device 100 or 200 is placed in a dark or poorly lit space, the photosensor will sense the light outside the housing 2 of the manicure device 100 or 200, obtain the lighting parameter from the light, and send the lighting parameter to the controller 6. If the lighting parameter corresponding to a dark or poorly lit space corresponds to the control procedure of turning on an additional light-emitting device of the manicure device 100 or 200, the controller 6 will, upon receiving and according to the lighting parameter, output the control procedure of turning on the light-emitting device in order for the light-emitting device to emit light and thereby increase the ambient light. If the lighting parameter corresponding to a dark or poorly lit space corresponds instead to adjusting the display device (not shown) of the manicure device 100 or 200 (e.g., adjusting the screen, backlight, press-key light, or indicator light of the display device), the controller 6 may, according to the lighting parameter, output the control procedure of adjusting the brightness of the display device. The lighting parameter corresponding to a dark or poorly lit space may also correspond to the control procedure of turning on a lighting device in the space (e.g., turning on a lamp in the room where the manicure device 100 or 200 is placed) so that upon receiving and according to such a lighting parameter, the controller 6 will output the control procedure of turning on the lighting device in the space. That is to say, the manicure device 100 or 200 can automatically control or operate a light source so as to be usable even in a poorly lit environment.

The lighting parameter includes color temperature, brightness, contrast or color rendering index; the present invention has no limitation in this regard.

The distance sensor is configured to sense a sensing signal and send out a signal, and the sensing signal and the signal correspond to a distance parameter. The distance sensor obtains the distance parameter by sensing the distance to a target object and sends the distance parameter to the controller 6, in order for the controller 6 to output a control procedure according to the distance parameter. The distance parameter may be a dynamically changing value. For example, in the course in which a user's hand approaches the upper housing portion 21 and then enters the internal cavity 3, the distance sensor senses the distance between the hand and the upper housing portion 21, the distance between the hand and the internal cavity 3, and the thickness and angle of appearance of each finger of the hand, and then sends the sensed distance parameters to the controller 6. Once the hand enters the internal cavity 3, and the distance parameter between the hand and the base portion 22 becomes 0 (i.e., when the hand is placed on the base portion 22) and is sensed and sent by the distance sensor to the controller 6, and if the distance parameter between the hand and the base portion 22 being 0 corresponds to the control procedure of turning on the UV light-emitting units 4, the controller 6 will turn on the UV light-emitting units 4 upon receiving and according to the distance parameter. Thus, the distance sensor enables the manicure device 100 or 200 to control or operate the UV light-emitting units 4 automatically. In another preferred embodiment, the distance sensor detects the distance between each two adjacent fingers (or more particularly each two adjacent fingernails) in the internal cavity 3 and sends the sensed distance parameters to the controller 6, in order for the controller 6 to calculate the position of and thereby precisely locate each finger. The controller 6 will then output a control procedure according to the distance parameters, wherein the control procedure may involve adjusting the UV light-emitting units 4 angularly toward the fingers, adjusting the position of the UV light-emitting units 4, issuing an alert to prompt the user to adjust the hand position, or adjusting curing condition (such as curing time or intensity of the light).

The distance sensor includes general devices that can be used to measure the distance between different objects, such as a linear displacement sensor, an angular displacement sensor, a Hall type displacement sensor, a conductive plastic displacement sensor, a metal glaze displacement sensor, a metal film displacement sensor, a magnetic-sensing displacement sensor, a photoelectric displacement sensor, a magnetostrictive displacement sensor, a digital laser displacement sensor, an eddy current displacement sensor, a triangle laser displacement sensor, a capacitive displacement sensor, a time difference laser displacement sensor, a white light confocal displacement sensor, a laser displacement sensor, a dispersion confocal displacement sensor, an optical fiber displacement sensor or a rotary displacement sensor; the present invention has no limitation in this regard. The term “target object” includes any object close to the environment of the manicure device 100 or 200. Preferably, the object includes any area of a hand(such as a fingernail, a finger, a palm or a wrist), any area of a foot (such as a toenail, a toe, a sole or an ankle) or a nail tip; the invention has no limitation in this regard.

The motion sensor is configured to sense a sensing signal and send out a signal, and the sensing signal and the signal correspond to an (angular) acceleration parameter, (angular) velocity parameter, or displacement parameter. More specifically, the motion sensor senses changes in the X-, Y-, and Z-axis directions to obtain the aforesaid parameter and thereby determine the moving or tilted state of the manicure device 100 or 200. The motion sensor obtains the acceleration parameter, velocity parameter, or displacement parameter by sensing motion changes in the environment and sends the acceleration parameter, velocity parameter, or displacement parameter to the controller 6, in order for the controller 6 to output a control procedure according to the acceleration parameter, velocity parameter, or displacement parameter. The term “motion changes in the environment” refers to the acceleration or angular acceleration of the manicure device 100 or 200 when the manicure device is being moved or tilted in the environment. For example, a technician may set a threshold value in advance, and the sensing signal exceeding the threshold value may correspond to the control procedure of turning off the manicure device 100 or 200 or the control procedure of issuing an alert so that when the manicure device 100 or 200 is moved and the motion sensor has sensed the acceleration parameter, velocity parameter, or displacement parameter of the manicure device 100 or 200 and sent the parameter to the controller 6, the controller 6 will, upon receiving and according to the sensing signal of the parameter, either output the control procedure of turning off the manicure device 100 or 200 and thereby turn off the manicure device 100 or 200, or output the control procedure of issuing an alert and thereby start the emission of an alerting sound or alerting light. The motion sensor can also be used to sense the way the manicure device 100 or 200 is placed, so a technician may further set a corresponding control procedure to prevent UV light leakage that may otherwise occur when the manicure device 100 or 200 is moved or improperly placed.

The magnetic field sensor is configured to sense a sensing signal and send out a signal, and the sensing signal and the signal correspond to a magnetic field parameter. The magnetic field sensor obtains the magnetic field parameter by sensing the state of the magnetic field in the environment and sends the magnetic field parameter to the controller 6, in order for the controller 6 to output a control procedure according to the magnetic field parameter. For example, when the magnetic field parameter of the environment sensed and sent by the magnetic field sensor to the controller 6 indicates a high chance of injury to the human body, especially user with medical devices (such as artificial pacemaker), and if the magnetic field parameter indicating injury to the human body corresponds to the control procedure of instructing the manicure device 100 or 200 to issue an alert, the controller 6 will, upon receiving and according to the magnetic field parameter, output the control procedure of instructing the manicure device 100 or 200 to issue an alert by a display device, sound-playing device, or light-emitting device and thereby notify the user that the manicure device 100 or 200 is in a magnetic field environment that is injurious to the human body; thus, the manicure device 100 or 200 has a safety protection device for notifying the user that the current operating environment may be harmful to the health of the human body, and providing suggestion to user to leave the operating environment and/or shut down the manicure device 100 or 200. In another preferred embodiment, an accessory (not shown) of the manicure device 100 or 200 has a magnetic field sensor, and the magnetic field sensor can obtain the magnetic field parameter by sensing the state of the magnetic field of the accessory and send the magnetic field parameter to the controller 6, in order for the controller 6 to determine according to the magnetic field parameter whether the accessory is properly oriented or positioned and, if necessary, output the control procedure of issuing an alert; that is to say, the magnetic field sensor and the controller can work together to make the installation of the accessory foolproof.

The magnetic field parameter includes, but is not limited to, current, magnetic force, and magnetic field.

The gyroscope is configured to sense a sensing signal and send out a signal, and the sensing signal and the signal correspond to an orientation parameter. The gyroscope obtains the orientation parameter by sensing the orientation of the environment and sends the orientation parameter to the controller 6, in order for the controller 6 to output a control procedure according to the orientation parameter. For example, when the manicure device 100 or 200 is not placed on a horizontal operating platform (e.g., tabletop) or in an acceptable orientation (e.g., when the operating platform is exceedingly skew, or when an obstacle has rendered the base portion 22 of the manicure device 100 or 200 askew), and if the orientation parameter corresponding to not being placed on a horizontal operating platform or in an acceptable orientation corresponds to the control procedure of turning off the manicure device 100 or 200, the controller 6 will, upon receiving and according to the orientation parameter of the manicure device sensed and sent by the gyroscope to the controller 6, output the control procedure of turning off the manicure device 100 or 200 and thereby turn off the manicure device 100 or 200. Thus, the gyroscope helps protect the manicure device 100 or 200 and prevent the manicure device 100 or 200 from being used on a skew operating platform, lest the photocuring effect of the manicure device be compromised or UV light leakage occur. Human eyes can be harmed or discomfort during the UV light leakage.

The GPS (Global Positioning System) device is configured to sense a sensing signal and send out a signal, and the sensing signal and the signal correspond to a geographic location parameter. The GPS device obtains the geographic location parameter by sensing the geographic location of the environment and sends the geographic location parameter to the controller 6, in order for the controller 6 to output a control procedure according to the geographic location parameter. For example, when the geographic location of the manicure device 100 or 200 is close to a market or store that sells manicure products, and if the geographic location parameter corresponding to being close to a market or store that sells manicure products corresponds to the control procedure of instructing the manicure device 100 or 200 to provide advertising information, the controller 6 will, upon receiving and according to the geographic location parameter of the manicure device 100 or 200 sensed and sent by the GPS device to the controller 6, output the control procedure of instructing the manicure device 100 or 200 to provide advertising information, in order for a display device, sound-playing device, or light-emitting device to issue an alert signal and thereby notify the user that the manicure device 100 or 200 is close to a market or store that sells manicure products. That is to say, the manicure device 100 or 200 may have an advertising function to help markets or stores to notify consumers that manicure products can be bought nearby. In another preferred embodiment, the geographic location parameter of the manicure device 100 or 200 is sensed by the GPS device, sent to the controller 6 by the GPS device, and then transmitted (e.g., wirelessly) by the controller 6 to a corporate database to facilitate big data analysis or logistic analysis at the market end.

The heart rate sensor is configured to sense a signal and send out a signal, and the sensing signal and the signal correspond to a heart rate parameter. The heart rate sensor obtains the heart rate parameter by sensing the heart rate of a living creature in the environment and sends the heart rate parameter to the controller 6, in order for the controller 6 to output a control procedure according to the heart rate parameter. For example, the heart rate sensor senses the heart rate parameter of a user's hand and sends the heart rate parameter to the controller 6. If an unhealthy heart rate parameter corresponds to the control procedure of instructing the manicure device 100 or 200 to issue an alert, the controller 6 will, upon receiving and according to such a heart rate parameter, output the control procedure of instructing the manicure device 100 or 200 to issue an alert, in order for a display device, sound-playing device, or light-emitting device to issue an alert signal and thereby notify the user of the manicure device 100 or 200 that he or she has an abnormal heart rate parameter and must pay attention to his or her body conditions. In other words, the heart rate sensor of the manicure device 100 or 200 can be used to monitor a user's heart rate and physical health.

The UV light sensor is configured to sense a sensing signal and send out a signal, and the sensing signal and the signal correspond to a UV light parameter. The UV light sensor obtains the UV light parameter by sensing the UV light in the environment and sends the UV light parameter to the controller 6, in order for the controller 6 to output a control procedure according to the UV light parameter. Preferably, the UV light sensor can sense the UV light parameter of the internal cavity 3, of a single one of the UV light-emitting units 4, or of the UV light-emitting units 4 in a certain region. For example, when the UV light intensity of the UV light-emitting units 4 is insufficient to cure a nail polish, or higher than required to cure the nail polish, or too high to be safe to the health of the human body, and if the UV light parameter corresponding to any of the above corresponds to the control procedure of instructing the manicure device 100 or 200 to issue an alert, the controller 6 will, upon receiving and according to the UV light parameter of the UV light-emitting units 4 sensed and sent by the UV light sensor to the controller 6, output the control procedure of instructing the manicure device 100 or 200 to issue an alert, in order for a display device, sound-playing device, or light-emitting device to issue an alert signal and thereby notify the user that the UV light in the internal cavity 3 needs adjustment or that the user must stop using the manicure device 100 or 200. The UV light parameter corresponding to an insufficient or excessively high UV light intensity may also correspond to the control procedure of automatically adjusting the UV light intensity of the UV light-emitting units 4 so that upon receiving and according to such a UV light parameter, the controller 6 will output the control procedure of automatically adjusting the UV light intensity of the UV light-emitting units 4 in order for the UV light-emitting units 4 to adjust their UV light intensity to a proper level automatically. Thus, the UV light sensor helps prevent the manicure device 100 or 200 from having an undesirable UV light intensity that may either damage the health of the human body or compromise the intended manicure effect.

The UV light sensor includes general devices that can be used to measure the UV light parameter, such as a gallium nitride UV light sensor, a photomultiplier tube, a silicon UV light sensor, a zinc oxide UV light sensor or an optical fiber UV light sensor; the present invention has no limitation in this regard.

The temperature sensor is configured to sense a sensing signal and send out a signal, and the sensing signal and the signal correspond to a temperature parameter. The temperature sensor obtains the temperature parameter by sensing a temperature (e.g., the temperature of the environment or of an object) and sends the temperature parameter to the controller 6, in order for the controller 6 to output a control procedure according to the temperature parameter. Preferably, the temperature sensor can sense the temperature parameter of the internal cavity 3. For example, if the temperature parameter of the internal cavity 3 is sensed by the temperature sensor as corresponding to an exceedingly high temperature, an abruptly changing temperature, or other abnormal temperature conditions and corresponds to the control procedure of instructing the manicure device 100 or 200 to issue an alert, the controller 6 will, upon receiving and according to the temperature parameter sent by the temperature sensor to the controller 6, output the control procedure of instructing the manicure device 100 or 200 to issue an alert, in order for a display device, sound-playing device, or light-emitting device to issue an alert signal and thereby notify the user to stop using the manicure device 100 or 200. The temperature parameter corresponding to an exceedingly high or abruptly changing temperature may also correspond to the control procedure of automatic temperature adjustment so that upon receiving and according to such a temperature parameter, the controller 6 will output the control procedure of automatic temperature adjustment in order for the manicure device 100 or 200 to adjust its temperature to a proper level automatically, e.g., to lower an excessively high temperature by dissipating heat through a heat dissipation device. Thus, the temperature sensor helps prevent the manicure device 100 or 200 from producing an abnormal (e.g., overly high or abruptly changing) temperature that may damage the health of the human body, compromise the intended manicure effect, or impair the manicure device 100 or 200 itself. In another preferred embodiment, the temperature sensor can sense the temperature parameter of an important object in the manicure device 100 or 200 and send the temperature parameter to the controller 6. If the temperature parameter corresponding to an overly high temperature of the important object corresponds to the control procedure of turning off the manicure device 100 or 200, the controller 6 will, upon receiving and according to such a temperature parameter, output the control procedure of turning off the manicure device 100 or 200 to protect the important object in the manicure device 100 or 200 from damage by high heat.

The temperature sensor includes general devices that can be used to measure temperature, such as a gas thermometer, a bimetallic strip thermometer, a hydraulic thermometer, a platinum resistance thermometer, a temperature-sensing semiconductor thermometer, a thermal resistance thermometer, a thermocouple thermometer, a transistor thermometer or an ultrasonic thermometer; the present invention has no limitation in this regard.

The chemical substance sensor is configured to sense a sensing signal and send out a signal, and the sensing signal and the signal correspond to a chemical substance parameter. The chemical substance sensor obtains the chemical substance parameter by sensing the chemical substance in the environment and sends the chemical substance parameter to the controller 6, in order for the controller 6 to output a control procedure according to the chemical substance parameter. For example, the chemical substance sensor senses the chemical substance parameter of the surroundings of the housing 2 or of the internal cavity 3 and sends the chemical substance parameter to the controller 6. When the manicure device 100 or 200 is in a particular chemical substance environment, e.g., an environment containing an excessive amount of volatile gas, and if the chemical substance parameter corresponding to an excessive amount of volatile gas corresponds to the control procedure of instructing the manicure device 100 or 200 to issue an alert, the controller 6 will, upon receiving and according to such a chemical substance parameter, output the control procedure of instructing the manicure device 100 or 200 to issue an alert, in order for a display device, sound-playing device, or light-emitting device to issue an alert signal and thereby notify the user that the current operating environment contains an excessive amount of volatile gas. Thus, the chemical substance sensor serves as a safety protection device of the manicure device 100 or 200 and can notify a user of the chemical substance state of the current operating environment. The chemical substance parameter can be a common chemical substance parameter, such as the amount of oxygen, the amount of volatile organic gas, or the amount of carbon dioxide; the present invention has no limitation in this regard. Preferably, the gas is a volatile organic gas, such as toluene or formaldehyde. The manicure device 100 or 200 can further has a fan or an air freshener in order to blow away or decrease gaseous chemical substances.

The chemical substance sensor includes general gas sensors that can be used to measure the above chemical substance parameters, such as an optical gas sensor, an infrared gas detector, a semiconductor gas sensor, a catalytic combustion gas sensor, a solid electrolyte gas sensor, an electrochemical gas sensor, a carbon dioxide sensor, an oxygen sensor, a volatile organic gas sensor or a formaldehyde gas sensor; the present invention has no limitation in this regard.

The radio frequency identification (RFID) device is configured to receive a sensing signal and/or transmit a signal, and the sensing signal and the signal correspond to an RFID parameter. In one preferred embodiment, the RFID device obtains the RFID parameter by receiving and/or transmitting an RFID parameter (e.g., a particular radio wave). For example, when the manicure device 100 or 200 carries the RFID device (e.g., an RFID tag), an external RFID reader can identify the RFID parameter of the RFID device and thereby obtain specific information. This RFID application is suitable for use in, among others, a warehousing or logistic management system to track the location and quantity of the manicure device 100 or 200. In another preferred embodiment, the RFID device obtains the RFID parameter by receiving and/or transmitting an RFID parameter and sends the RFID parameter to the controller 6, in order for the controller 6 to output a control procedure according to the RFID parameter. For example, when a user carries an RFID tag, the RFID device (e.g., an RFID reader) of the manicure device 100 or 200 can sense the RFID parameter of the RFID tag and sends the RFID parameter to the controller 6. When the RFID tag on the user is provided with an RFID parameter corresponding to the user's previous use mode or the use mode to be set, and if the RFID parameter corresponding to the user's previous use mode or the use mode to be set corresponds to the control procedure of activating the user's previous use mode or to-be-set use mode, the controller 6 will, upon receiving and according to the RFID parameter, output the control procedure of activating the user's previous use mode or to-be-set use mode and thereby activate the manicure device 100 or 200 in the user's previous or to-be-set use mode. In addition, various types or brands of gel nail polishes can have its own RFID tag. When the RFID tag of the gel nail polishes is provided with an RFID parameter corresponding to the best mode for applying the gel nail polishes, the controller 6 will, upon receiving and according to the RFID parameter, output the control procedure of activating the best mode for applying the gel nail polishes and thereby activate the manicure device 100 or 200 in the best mode for applying the gel nail polishes. Moreover, the control procedure may involve providing suggestion to the user how to decorate the nail, such as painting the nails with at least two or more layers of gel nail polishes, or no painting of gel nail polishes on the lateral side of the nail; the present invention has no limitation in this regard. Thus, the RFID device helps control the manicure device 100 or 200 to work in a preset working mode automatically. Or, when the RFID tag on the user is provided with an RFID parameter by which the user can be identified and which corresponds to the control procedure of logging in the user, the controller 6 will, upon receiving and according to the RFID parameter, output the control procedure of identifying and logging in the user and turn on the manicure device 100 or 200, with the manicure device operating in the user's preferred use mode, showing a welcome screen image, etc.

The dust sensor is configured to sense a sensing signal and send out a signal, and the sensing signal and the signal correspond to a dust parameter. The dust sensor obtains the dust parameter by sensing the dust in the environment and sends the dust parameter to the controller 6, in order for the controller 6 to output a control procedure according to the dust parameter. For example, the dust sensor senses the dust parameter of the surroundings of the housing 2 or of the internal cavity 3 and sends the dust parameter to the controller 6. If the dust parameter exceeds a standard value and indicates harm to the human body or has a negative effect on nail painting with the manicure device 100 or 200, and if the control procedure corresponding to this dust parameter is to instruct the manicure device 100 or 200 to issue an alert, the controller 6 will, upon receiving and according to the dust parameter, output the control procedure of instructing the manicure device 100 or 200 to issue an alert, in order for a display device, sound-playing device, or light-emitting device to issue an alert signal and thereby notify the user that the dust parameter of the current operating environment has exceeded the standard value and may compromise the intended manicure effect or damage the health of the user's body. A dust parameter that exceeds the standard value and may compromise the intended manicure effect may also correspond to the control procedure of turning off the manicure device 100 or 200 automatically so that upon receiving and according to such a dust parameter, the controller 6 will output the control procedure of turning off the manicure device 100 or 200 automatically and thereby turn off the manicure device 100 or 200 automatically, lest the manicure device 100 or 200 be overheated because of dust. Thus, the dust sensor helps prevent accumulated dust from affecting the operation of the manicure device 100 or 200 or compromising the intended manicure effect. The manicure device 100 or 200 can further has a dust collector in order to clean up the dust.

The dust parameter includes but is not limited to dust, smoke, mine dust, sand or powder.

The dust sensor includes any general sensor for sensing the above dust parameters, such as a PM2.5 sensor, a laser dust sensor, a laser PM 2.5 dust sensor, an optical dust sensor, a digital dust concentration sensor; the present invention has no limitation in this regard.

The position detector is configured to sense a sensing signal and send out a signal, and the sensing signal and the signal correspond to a position parameter and may be an infrared sensor, an ultrasound sensor, a Doppler radar sensor, or the like, without limitation. The position detector obtains the position parameter by sensing the state of an object in an area of interest and sends the position parameter to the controller 6, in order for the controller 6 to output a control procedure according to the position parameter. The position detector may also be a virtual image key sensor, which senses a sensing signal corresponding to a virtual image key parameter. The virtual image key sensor obtains the virtual image key parameter by sensing a virtual image key in the environment and/or an altered image of the virtual image key and sends the virtual image key parameter to the controller 6, in order for the controller 6 to output a control procedure according to the virtual image key parameter. For example, a virtual image key for the UV light-emitting units 4 is projected in front of the housing of the manicure device 100 or 200, and the virtual image key sensor senses the virtual image key being touched (i.e., the projected image of the virtual image key being altered by the finger touching it) as the virtual image key parameter. If this virtual image key parameter corresponds to the control procedure of activating the UV light-emitting units 4, the controller 6 will, upon receiving and according to the virtual image key parameter, output the control procedure of activating the UV light-emitting units 4 and thereby activate the UV light-emitting units 4. Thus, the virtual image key sensor enables the manicure device 100 or 200 to be controlled with a virtual image key.

The virtual image key may be the virtual image key(s) generated by any image projector, such as the virtual image key(s) (e.g., the image of a press-button or a keyboard) generated by a laser or other optical projection device; the present invention has no limitation in this regard. The virtual image key may be generated by an image projector provided in the manicure device 100 or 200 or by an external device. The virtual image key saves the space otherwise required for a common physical press-key. More specifically, the virtual image key does not occupy the space on the housing 2 of the manicure device as does its physical counterpart, and this is especially advantageous when multiple control function keys are needed. Furthermore, the virtual image key will not come off or have contact problems after long-term use, and this contributes to extending the service life of the manicure device 100 or 200.

The term “altered image of the virtual image key” refers to any alteration of the image of the virtual image key, such as the image of the virtual image key becoming askew when touched by a finger.

The virtual image key parameter includes a signal corresponding to the virtual image key and/or an altered image of the virtual image key.

Preferably, the manicure device 100 or 200 is provided with the virtual image key sensor, and the virtual image key sensor can supersede the input interface 7, i.e., a user can input a setting instruction directly through the virtual image key.

The infrared sensor of the present invention is configured to sense an object that enters into the cavity. In one embodiment, the infrared sensor senses the object by means of sensing the heat from the object. For example, when user's hand enters into the cavity, the infrared sensor will sense the heat from the user's hand and then the controller 6 will output a control procedure, such as turn the manicure device 100 or 200 into operating mode and/or start up the UV light-emitting units 4. In another embodiment, the infrared sensor includes an infrared light emitting unit and an infrared light receiving unit. The infrared sensor senses the object by means of that the infrared light receiving unit stops receiving infrared light from the infrared light emitting unit, or the infrared light receiving unit starts to receive reflected infrared light from the object. For example, the infrared light emitting unit keeps emitting infrared light to the infrared light receiving unit when the manicure device 100 or 200 is in standby mode. When user's hand enters into the cavity, the infrared light from the infrared light emitting unit will be blocked by user's hand so that the infrared light receiving unit stops receiving infrared light from the infrared light emitting unit, and the controller 6 will output a control procedure (such as the aforementioned control procedure). In another example, the infrared light emitting unit is configured next to the infrared light receiving unit. When user's hand enters into the cavity, the infrared light from the infrared light emitting unit will be emitted to the user's hand and then reflected to the infrared light receiving unit next to the infrared light emitting unit. When the infrared light receiving unit starts to receive the reflected infrared light from the user's hand, the controller 6 will output a control procedure (such as the aforementioned control procedure).

Therefore, the manicure device of the present invention has at least one sensor to make it easier for a user to know the working mode and operating conditions of the manicure device. Moreover, the manicure device of the invention can operate under manual or automatic control in order to have its working mode and operating conditions adjusted or set, or to issue a notification signal.

What is claimed is:

1. A manicure device, comprising:
a manicure device body including:
a housing,
an internal cavity provided at one side of the housing and forms an opening at the housing, and
at least one ultraviolet (UV) light-emitting unit provided in the housing and is configured to emit light toward the internal cavity;
at least one sensor provided at the housing, wherein the sensor sends out a signal when triggered; and
a controller provided on or in the housing, wherein the controller is connected to the sensor and is configured to receive the signal of the sensor.

2. The manicure device of claim 1, wherein the manicure device further includes an input interface configured to generate a setting signal through inputting, and the setting signal is sent to the controller, in order for the controller to perform a control procedure according to the setting signal.

3. The manicure device of claim 2, wherein the sensor includes a hand gesture sensor, a photosensor, a distance sensor, an motion sensor, a magnetic field sensor, a gyroscope, a GPS (Global Positioning System) device, a heart rate sensor, a UV light sensor, a temperature sensor, a chemical substance sensor, a radio frequency identification (RFID) device, a dust sensor, a position detector a virtual image key sensor or an infrared sensor.

4. The manicure device of claim 1, wherein the sensor is a hand gesture sensor, the signal corresponds to a hand gesture feature, and the hand gesture sensor sends the hand gesture feature to the controller, in order for the controller to output a control procedure according to the hand gesture feature.

5. The manicure device of claim 3, wherein the hand gesture sensor is an ultrasound sensing device or an infrared sensing device.

6. The manicure device of claim 4, wherein the hand gesture sensor is an ultrasound sensing device or an infrared sensing device.

7. The manicure device of claim 1, wherein the sensor is a photosensor, the signal corresponds to a lighting parameter, and the photosensor sends the lighting parameter to the controller, in order for the controller to output a control procedure according to the lighting parameter.

8. The manicure device of claim 1, wherein the sensor is a distance sensor, the signal corresponds to a distance parameter, and the distance sensor sends the distance parameter to the controller, in order for the controller to output a control procedure according to the distance parameter.

9. The manicure device of claim 1, wherein the sensor is an motion sensor, the signal corresponds to an acceleration parameter, velocity parameter, or displacement parameter, and the motion sensor sends the acceleration parameter, velocity parameter, or displacement parameter to the controller, in order for the controller to output a control procedure according to the acceleration parameter, velocity parameter, or displacement parameter.

10. The manicure device of claim 1, wherein the sensor is a magnetic field sensor, the signal corresponds to a magnetic field parameter, and the magnetic field sensor sends the magnetic field parameter to the controller, in order for the controller to output a control procedure according to the magnetic field parameter.

11. The manicure device of claim 1, wherein the sensor is a gyroscope, the signal corresponds to an orientation parameter, and the gyroscope sends the orientation parameter to the controller, in order for the controller to output a control procedure according to the orientation parameter.

12. The manicure device of claim 1, wherein the sensor is a GPS (Global Positioning System) device, the signal corresponds to a geographic location parameter, and the GPS device sends the geographic location parameter to the controller, in order for the controller to output a control procedure according to the geographic location parameter.

13. The manicure device of claim 1, wherein the sensor is a heart rate sensor, the signal corresponds to a heart rate parameter, and the heart rate sensor sends the heart rate parameter to the controller, in order for the controller to output a control procedure according to the heart rate parameter.

14. The manicure device of claim 1, wherein the sensor is a UV light sensor, the signal corresponds to a UV light parameter, and the UV light sensor sends the UV light parameter to the controller, in order for the controller to output a control procedure according to the UV light parameter.

15. The manicure device of claim 1, wherein the sensor is a temperature sensor, the signal corresponds to a temperature parameter, and the temperature sensor sends the temperature parameter to the controller, in order for the controller to output a control procedure according to the temperature parameter.

16. The manicure device of claim 1, wherein the sensor is a chemical substance sensor, the signal corresponds to a chemical substance parameter, and the chemical substance sensor sends the chemical substance parameter to the controller, in order for the controller to output a control procedure according to the chemical substance parameter.

17. The manicure device of claim 1, wherein the sensor is a radio frequency identification (RFID) device, the signal corresponds to an RFID parameter, and the RFID device sends the RFID parameter to the controller, in order for the controller to output a control procedure according to the RFID parameter; and/or, an external RFID reader can identify RFID parameter of the RFID device in order for the external RFID reader to obtain specific information and/or output corresponding procedure according to the RFID parameter of the RFID device.

19. The manicure device of claim 1, wherein the sensor is a dust sensor, the signal corresponds to a dust parameter, and the dust sensor sends the dust parameter to the controller, in order for the controller to output a control procedure according to the dust parameter.

20. The manicure device of claim 1, wherein the sensor is a position detector, the signal corresponds to a position parameter, and the position detector sends the position parameter to the controller, in order for the controller to output a control procedure according to the position parameter.

21. The manicure device of claim 20, wherein the position detector is a virtual image key sensor, the signal corresponds to a virtual image key parameter, and the virtual image key sensor sends the virtual image key parameter to the controller, in order for the controller to output a control procedure according to the virtual image key parameter.

22. The manicure device of claim 21, wherein the virtual image key is generated by an image projector.

23. The manicure device of claim 1, wherein each of the ultraviolet (UV) light-emitting unit comprises at least one UV LED chip, and the UV LED chip has a full width half maximum equal or smaller than 30 nm.

24. The manicure device of claim 1, wherein the power consumption of the manicure device is smaller than 40 watts.

25. The manicure device of claim 1, further comprises an image analysis module provided in an interior of the housing, wherein the image analysis module comprises a camera unit and an analysis unit, the camera unit shots a picture of an object, the analysis unit analysis the picture and sends result parameter to the controller, in order for the controller to output a control procedure according to the result parameter.
